(19) United States
(12) Patent Application Publication
(10) Pub. No.: US 2004/0198770 A1
(43) Pub. Date: Oct. 7, 2004

(54) Uses for l-tetrahydropalmatine

(75) Inventor: Hong Xue, Hong Kong (CN)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: PharmacoGenetics Limited, Kowloon (HK)

(21) Appl. No.: 10/406,811

(22) Filed: Apr. 3, 2003

(51) Int. Cl. A61K 31/4745

(52) U.S. Cl. 514/306

(57) ABSTRACT

The present invention concerns novel uses of l-tetrahydropalmatine (l-THP) and its related compounds, methods of treatment of patients in need of same, and methods of manufacture of medicaments for treatment of patients, and the use of l-THP in same. Specifically, it is shown herein that l-THP has an anxiolytic effect (i.e., that it is therapeutically effective in relieving or reducing anxiety, agitation and/or tension), and that it has a sedative effect depending on its quantity of oral administration.

### BACKGROUND OF THE INVENTION

Corydalis yanhusuo W. T. Wang is a well-known traditional Chinese medicine which has been used to alleviate pain such as headache, chest pain, hypochondriac pain, epigastric pain, abdominal pain, backache, arthralgia, dysmenorrhea or trauma (Handbook of the composition and pharmacology of common Chinese drugs, In: Huang T K, editor. Beijing: Chung-kuo i yao ko chi chu pan she, 1994: 874-881). dl-THP (also known as Corydalis B, full name 5,8,18,13a-tetrahydro-2,3,9,10-tetramethoxy-6H-dibenzo [a,q] quinolizine) is one of the active constituents isolated from Corydalis yanhusuo W. T. Wang which has in the past been shown to have a number of therapeutic effects.

dl-THP has been shown to deplete the levels of dopamine, noradrenaline and serotonin in the CNS (Liu GQ et al., Arch Int Pharmacodyn Ther 1982 July258(1): 39-50; PMID 6182845), and to decrease both arterial pressure and heart rate through a serotonergic release process in the hypothalamus (Chueh F Y et al., Jpn J Pharmacol. October 1995; 69(2): 177-80; PMID: 8569056). It also decreases motor activity. It is also known to be protective in rat heatstrokes (Chang C K et al., Neurosci Lett. May 28, 1999; 267(2): 109-12; PMID: 10400224).

In recent years, it has been discovered that dl-THP has anxiolytic effects such that its therapeutic effect would be diverse while taking proprietary dosages. This discovery has been described in U.S. Pat. No. 6,521,634 B2, issued on Feb. 18, 2003. The entire teachings of the application are incorporated herein by reference.

Targets in the CNS for the two enantiomers of dl-THP have been identified and therapeutic effects shown, including causing a sedative-tranquillising effect and inhibiting voltage-dependent Ca²⁺ channels (Vauquelin et al., Neurochemistry International, 1989 15(3): 321-324). The tranquillising action of l-THP has been considered related to the blocking of the DA receptor.

l-THP is widely available and is sold as a herbal dietary supplement and as an analgesic and sleeping pill. In China, both chemical medicine called Rotundine (contains 30-60 mg of l-THP per tablet) and proprietary Chinese medicine called Complex Formula Date Seed (“Fu Fang Zao Ren”) Capsule (contains 60 mg l-THP per capsule) are SDA (State Drug Administration, PRC) approved OTC (Over-the-counter) drugs for its analgesic, sedative and hypnotic properties.

l-THP can be isolated from Chinese herbs such as Stephania for clinical use using standard techniques well known in the art (see for example Matsuda H. et al., “Inhibitory effects of methanolic extract from corydalis tuber against types I-IV allergic models.”, Biol Pharm Bull. 1995 July; 18(7): 963-7; PMID: 7581251), as well as synthesised using standard techniques (Narasimhan N. S. et al., “A novel synthesis of tetrahydropalmatine.”, Chem. Ind. May 10, 1969; 19: 621-2; PMID: 5781510).

### SUMMARY OF THE INVENTION

According to the present invention, a method of manufacture of a composition (e.g., a medicament), comprising l-THP (or one or more of its related compounds) and a physiologically acceptable carrier, for the treatment of anxiety disorders is provided. This contrasts with previously reported effects of l-THP such as its sedative-tranquillising effect. In recent years, anxiety disorders have been classified for clinical diagnosis. In general, they include panic disorder, social phobia, obsessive-compulsive disorder, trauma stress disorder, acute stress disorder, generalized anxiety disorder and specific phobia. (Charney D. S. et al., “Neurobiology of Mental Illness.” Oxford University Press. 1999; 442-8).

Also provided is a method of treatment of anxiety disorders as defined above in a patient, comprising administering to the said patient a therapeutically effective quantity of l-THP.

### DETAILED DESCRIPTION OF THE INVENTION

A description of preferred embodiments of the invention follows.

GABA (gamma-aminobutyric acid) is regarded as one of the major inhibitory amino acid transmitters in the central nervous system (CNS) of the mammalian brain. GABA is synthesized from glutamic acid, the major excitatory neurotransmitter, by one of two forms of glutamic acid decarboxylase (GAD). About 30% of neurons in the brain, particularly small interncurons, are thought to be GABAergic (contain GAD), and most neurons will respond to GABA by reducing their firing rate. They are widely, although unequally, distributed through the mammalian brain. An enormous amount of effort has been devoted to implicating GABA in the etiology of anxiety, seizure disorder, sleep disorder and cognition (Tallman J. F. et al., “The GABA-ergic system: a locus of benzodiazepine action.”, Annu Rev Neurosci. 1985; 8: 21-44; PMID: 2858999). GABA mediates many of its actions through GABA receptors localized both on cell bodies and on nerve endings. Postsynaptic responses to GABA are mediated through alterations in chloride conductance that generally lead to hyperpolarization of the cell. Recent research has found that the complex of proteins associated with postsynaptic GABA responses is a major site of action for a number of structurally unrelated compounds capable of modifying postsynaptic responses to GABA. Depending on the mode of interaction, these compounds are capable of producing a spectrum of effects, such as sedative, anticonvulsant and anxiolytic, or wakefulness, seizures and anxiety.

The GABA_{A} receptor has a number of functional domains (Smith G. B., Olsen R. W., Trends Pharmacol Sci. 1995 May; 16(5):162-8; PMID: 7624971) and has located, in or near its chloride ion channel, a number of binding sites for benzodiazepines, barbiturates and picrotoxins, as well as sites for the anaesthetic steroids. In particular, the gamma subunit appears to enable drugs like benzodiazepines to modify the GABA responses (Pritchett D. B. et al., Nature, Apr. 13, 1989; 338(6216): 582-5; PMID: 2538761).

The class of benzodiazepines includes diazepam, trizolam and flunitrazepam. The principal behavioural effects of classical benzodiazepines in animals are four-fold: relief of anxiety, anticonvulsant effects, sedation and myorelaxation. These properties are shared by all full benzodiazepine agonists, regardless of the therapeutic indication for which they are prescribed. For instance, trizolam, prescribed as a hypnotic, is also a potent anxiolytic and anticonvulsant in animal tests, whereas diazepam, prescribed principally as an anxiolytic, is a powerful hypnotic in animals. It can be considered that all full agonists from other chemical series have equivalent behavioural effects. All these effects are blocked by benzodiazepine antagonists, indicating that they are indeed mediated by a direct interaction with the GABA_{A} receptor.

Drugs that interact at the BDZ binding site of the GABA_{A} receptor can possess a spectrum of pharmacological activities depending on their abilities to modify the actions of GABA. Those compounds that bind to the receptor and which possess activity similar to that of the BDZs are called agonists. Compounds that bind to the receptor and which possess activity opposite to that of the BDZs are called inverse agonists, and compounds which block both types of activity are termed antagonists.

When GABA binds to a GABA_{A} receptor, the chloride ion flux through the channel is increased. This leads to membrane hyperpolarization that results in a reduction in the excitability potential of the neuron. Consequently, GABA_{A} receptors are the molecular targets of a variety of pharmacologically and clinically important drugs, such as the anxiolytic, anticonvulsant, sedative-hypnotic BDZs, some anxiogenic, convulsant β-carbolines, and the convulsants bicuculline or picrotoxin.

Thus the use of l-THP in the present invention effects a response from the GABA_{A} receptor. In particular, l-THP can be used for the treatment of CNS disorders including the treatment of anxiety. The sedative/hypnotic properties of l-THP have been previously disclosed. The property of being an anxiolytic has not been previously suggested for l-THP. As used herein, the term “treatment” refers to any means designed to cure, alleviate, remove or lessen the symptoms of, or prevent or reduce the possibility of contracting any disorder or malfunction of the human or animal body.

The experiments below demonstrate the administration of therapeutically effective quantities of l-THP to mice. It can readily be administered to other mammals to achieve the same therapeutic effects, for example, but not limited to, humans, canines and felines as well as other domesticated animals and e.g., bovines and equines. l-THP can be formulated with a physiologically acceptable carrier, diluent or excipient (Remington's Pharmaceutical Sciences and US Pharmacopoeia, 1984, Mack Publishing Company, Easton, Pa., USA; United States Pharmacopoeia, ISBN: 1889788031). Reference herein to physiologically acceptable carriers is also reference to physiologically acceptable diluents and excipients as appropriate.

The medicinal effect of l-THP depends on the dosage administered. As the dosage of l-THP is increased, it changes from anxiolytic to sedative and anaesthetic. Under proprietary low dosage (for the experimental mice, 0.1-2.5 mg/kg, Optimal 1 mg/kg), l-THP has anxiolytic effect; under medium dosage (for the experimental mice, 5 mg/kg), l-THP has sedative and hypnotic effect; and under high dosage (20 mg/kg) has inhibitive effect on CNS and myorelaxation.

Exact dosages for a given therapeutic effect are dependent upon a number of factors, particularly the age, weight and sex of the patient to whom the composition is to be administered. Optimal dosages for a given therapeutic effect are determined using simple dose-response assays.

Exemplification

The experiments below show that l-THP has an anxiolytic effect (i.e., that it is therapeutically effective in relieving or reducing anxiety, agitation and/or tension). They also show that it has a sedative effect depending on its quantity of oral administration.

Animals

ICR mice of male sex, weighing 15.6-17.8 g were used, provided by Experimental Animal Centre of HKUST, housed in groups, given food and water ad libitum and maintained on a 12 hour light: 12 hour dark cycle. All of the experimental groups had 12 animals per group.

Drugs

(1) l-THP, purity ≧98%, was dissolved in 1% sulphuric acid solvent for test;

(2) Diazepam, provided by Sigma, was dissolved in 1% sulphuric acid solvent for test;

(3) dl-THP, purity ≧98%, was dissolved in 1% sulphuric acid solvent for test.

Diazepam and dl-THP are controls for I-THP.

Group and Dosage

(1) For l-THP groups, dosage was 0.05 mg/kg; 0.1 mg/kg; 0.5 mg/kg; 1.0 mg/kg; 2.5 mg/kg; 5 mg/kg; 10 mg/kg; 20 mg/kg; 30 mg/kg and 40 mg/kg, respectively;

(2) For dl-THP groups, dosage was 0.1 mg/kg; 0.5 mg/kg; 1.0 mg/kg and 5 mg/kg, respectively;

(3) For Diazepam group, dosage was 1 mg/kg;

(4) For normal control group, double-distilled water was used as the vehicle.

For each experimental animal, administered orally 0.2 ml (pH 7.2) of respective solvent at concentrations as stated above for each group (or double-distilled water for normal control group) one hour before testing.

Statistics

The test results are expressed as mean ± standard error of mean (X±S). Data of individual treatments and controls are analysed in percentage. Post hoc comparisons between data of groups are made using t-test and X² test.

Experimental Condition

All procedures were carried out in a quiet, air-conditioned laboratory at ambient temperature of 20-22° C. At the end of each session, boluses were removed and the box was thoroughly wiped with 70% ethanol.

Locomotor Activity Test

An apparatus with a brown light-resistant box having dimensions of 60×60×30 cm was used to perform the test. It consists of four cylindrical plastic boxes of 20 cm diameter, each having 6 equally distributed infrared photocells. The locomotor activity was counted automatically during a 300 second test period. A decrease in the number of transitions reflects a decrease in locomotor activity.

Horizontal-Wire Test

Mice are lifted by the tail and allowed to grasp a horizontally strung wire (1 mm diameter, 20 cm long and placed 20 cm above the table) with their forepaws and released (Bonetti E. P. et al., Psychopharmacology (Berl). 1982; 78(1): 8-18; PMID: 6292984). For each experimental animal, the number of falling onto the table in 60 seconds was determined. The results in Table 1 show that the number of falling increased in the l-THP 20 mg/kg and 40 mg/kg dosage groups compared to low dosage groups and normal control group.

Table 1 demonstrates the performance of mice after oral administration of l-THP both in the locomotor test and horizontal wire test. The locomotor activities of 20 mg/kg and 40 mg/kg group decreased significantly compared to low dosage groups and normal control group. These dosage groups exhibited symptoms of sleepiness and a reduced response to external stimulation. Although they could be stimulated to wake up, the transitions decreased significantly. Physical reflections existed for this group and there was no abnormal phenomenon in respiratory circulation. The 0.5-10 mg/kg groups showed no significant difference compared to the normal control group.

Table 1 also shows that l-THP inhibits the central nervous system (CNS) and muscle activities when the dosage of l-THP is higher than 20 mg/kg. This inhibition is positively related to dose administration.

TABLE 1

| Group | | No. of Transitions | No. of falling | Inducing sleep |
|---|---|---|---|---|
| 0.5 | mg/kg | 121.1 ± 9.53 | 0.4 ± 0.49 | — |
| 1.0 | mg/kg | 111.3 ± 11.91 | 0.4 ± 0.48 | — |
| 5.0 | mg/kg | 104.5 ± 10.73* | 0.5 ± 0.67* | — |
| 10 | mg/kg | 101.4 ± 15.41* | 1.0 ± 0.77* | — |
| 20 | mg/kg | 53.0 ± 12.72* | 4.5 ± 1.50* | + |
| 40 | mg/kg | 13.6 ± 5.27* | 8.5 ± 3.55* | ++ |

| | | | |
|---|---|---|---|
| Control | 119.5 ± 10.81 | 0.4 ± 0.49 | — |

n = 13
*p < 0.001

Table 2 shows the comparison of muscle strength (number of falling) of mice between l-THP and dl-THP of same dosages in the horizontal-wire test. There is no significant difference between same dosage l-THP and dl-THP groups. The number of falling significantly increases when the dosages of l-THP and dl-THP are greater than 30 mg/kg. Review to Table 1, dosage of 20 mg/kg of l-THP has already showed the effect of myorelaxation, so that it can be concluded that for l-THP, when the oral dosage is less than 20 mg/kg, there is no myorelaxation effect, but when it is greater than 20 mg/kg, reduction of muscle strength is induced.

TABLE 2
Number of falling

| | | | |
|---|---|---|---|
| Group | l-THP | dl-THP | p value |

| | | | | |
|---|---|---|---|---|
| 0.5 | mg/kg | 0.40 ± 0.48 | 0.30 ± 0.48 | >0.05 |
| 1.0 | mg/kg | 0.40 ± 0.48 | 0.50 ± 0.67 | >0.05 |
| 5.0 | mg/kg | 0.50 ± 0.67 | 0.40 ± 0.49 | >0.05 |
| 10 | mg/kg | 1.00 ± 0.77 | 0.80 ± 0.64 | >0.05 |
| 30 | mg/kg | 18.50 ± 3.54** | 21.60 ± 8.72** | >0.05 |

| | |
|---|---|
| Control | 0.4 ± 0.48 |

n = 12

Elevated Plus-Maze Test

The elevated plus-maze is made of 0.5 cm thick wood as a horizontal cross consisting of two open arms (25×5 cm) and two opposite arms (25×5 cm) enclosed by 10 cm high walls. The arms extend from a central platform having dimensions of 5×5 cm. The plus-maze is elevated to a height of 40 cm from the floor. The maze is put inside a box with dimensions of 30×30×50 cm. The mice are placed on the central platform of the maze facing a closed arm. The number of arm entries and the time spent in the open and closed arm are counted for 300 seconds (Pellow S. et al., “Anxiolytic and anxiogenic drug effects on exploratory activity in an elevated plus-maze: a novel test of anxiety in the rat”, Pharmacol Biochem Behav. 1986 March; 24(3): 525-9; PMID: 2871560). Arm entry was defined as one half body entered into the arm. A selective increase in the parameters corresponding to open arms reveals an anxiolytic effect.

The results in Table 3 show the number and percentage of mice entry after oral administration of l-THP in the elevated plus-maze test. The number and percentage of open arm entry increases when the oral dosage of l-THP is between about 0.1-2.5 mg/kg. The effect at 1.0 mg/kg is the most obvious. Compared to the normal control group, the percentage of open arm entry increases by 39.31-48.14%, p<0.05-0.01. Nevertheless, none of the l-THP dosage groups surpassed the level of the Diazepam (DZ) 1 mg/kg group.

TABLE 3

| | | | Open arm entry | | Closed arm entry | |
|---|---|---|---|---|---|---|
| Group | | Total no. | No. | % | No. | % |
| 0.05 | mg/kg | 28.08 ± 5.62 | 8.75 ± 1.36 | 32.11 ± 6.77 | 19.33 ± 5.55 | 67.89 ± 6.77 |
| 0.1 | mg/kg | 26.82 ± 4.38 | 11.55 ± 2.50* | 42.83 ± 4.26* | 15.27 ± 2.41 | 57.17 ± 4.26 |
| 0.5 | mg/kg | 27.92 ± 4.08 | 11.75 ± 3.14* | 41.82 ± 6.37* | 16.17 ± 1.95 | 58.18 ± 6.37 |
| 1.0 | mg/kg | 26.92 ± 3.61 | 12.00 ± 2.00* | 44.47 ± 3.10* | 14.92 ± 1.93 | 55.53 ± 3.10 |
| 2.5 | mg/kg | 26.83 ± 4.20 | 10.92 ± 2.50* | 40.39 ± 4.09* | 15.92 ± 2.15 | 59.61 ± 4.06 |
| 5.0 | mg/kg | 11.17 ± 5.13* | 3.33 ± 1.92* | 29.63 ± 5.03 | 7.83 ± 3.43 | 70.37 ± 5.03 |
| DZ 1 | mg/kg | 38.25 ± 7.06 | 21.50 ± 4.52* | 55.42 ± 4.10* | 16.75 ± 3.26 | 44.58 ± 4.10 |

| | | | | | |
|---|---|---|---|---|---|
| Control | 26.43 ± 3.41 | 8.00 ± 1.84 | 30.02 ± 4.24 | 18.43 ± 2.10 | 69.98 ± 4.24 |

n = 12
Compared to normal control group
*p < 0.001

Table 3 also shows that the activities of most of the experimental mice decrease significantly when the oral dosage was 5 mg/kg. Some mice were sleepy, had a reduced response to external stimulation, and exhibited the symptom of nervous inhibition.

Table 4 shows the time spent and percentage of mice entry after oral administration of l-THP in the elevated plus-maze test. The time spent in the open arm entry significantly increased when the oral dosage of l-THP is 0.1-2.5 mg/kg and the effect at 1.0 mg/kg is the most obvious. Compared to the normal control group, the percentage increases by 27.19-50.78%, p<0.05-0.01. The data of all dosages of l-THP are significantly lower than the level of Diazepam control group.

When the oral dosage is 5 mg/kg, the time spent in the open arm is obviously lower than the level of normal control group and most of the mice were drowsy or falling asleep in the closed arm entry.

TABLE 4

| | | Open arm | | Closed arm | |
|---|---|---|---|---|---|
| Group | | Time spent | % | Time spent | % |
| 0.05 | mg/kg | 53.08 ± 10.40 | 19.04 ± 3.55 | 225.42 ± 12.13 | 80.96 ± 3.55 |
| 0.1 | mg/kg | 68.18 ± 20.68 | 23.67 ± 5.61 | 217.27 ± 15.11 | 76.33 ± 5.61 |
| 0.5 | mg/kg | 71.75 ± 21.08 | 25.81 ± 7.09* | 205.33 ± 20.02 | 74.19 ± 7.09 |
| 1.0 | mg/kg | 80.08 ± 21.03* | 28.06 ± 6.70* | 204.00 ± 16.42 | 71.94 ± 6.70 |
| 2.5 | mg/kg | 64.75 ± 16.70* | 21.96 ± 4.70 | 227.83 ± 12.15 | 78.04 ± 4.70 |
| 5.0 | mg/kg | 32.33 ± 14.43* | 11.46 ± 5.48* | 253.25 ± 24.32 | 88.54 ± 5.48 |
| DZ 1 | mg/kg | 131.93 ± 19.42* | 45.19 ± 4.53* | 159.29 ± 14.18* | 54.81 ± 4.53* |

| | | | | |
|---|---|---|---|---|
| Control | 52.07 ± 8.74 | 18.61 ± 3.46 | 229.07 ± 19.39 | 81.39 ± 4.24 |

n = 12
*p < 0.01

The results in Table 5 compare the number and percentage of total and open arm entry of mice between l-THP and dl-THP in the elevated plus-maze test. Both l-THP & dl-THP increase the number and percentage of open arm entry. For 0.1 mg/kg group, the number and percentage of open arm entry of l-THP is greater than dl-THP by 31.7% and 40.4%, p<0.05. For 5 mg/kg group, the number and percentage of open arm entry of l-THP is less than dl-THP by 357.4% and 69.5%, p<0.01. Again, the activities of the l-THP 5 mg/kg dosage group were significantly reduced and most of the mice were drowsy and fell asleep in the closed arm entry. The dl-THP 5 mg/kg dosage group are not sleepy and do not exhibit a reduced response to external stimuli demonstrating that 5 mg/kg dl-THP does not induce a hypnotic effect or inhibit the nervous system. The medicinal efficacy both of l-THP & dl-THP groups is lower than DZ 1 mg/kg group but obviously higher than normal control group with significant statistical difference.

TABLE 5

| | | Total no. | | Open arm entry no. | | % | |
|---|---|---|---|---|---|---|---|
| Group | | l-THP | dl-THP | l-THP | dl-THP | l-THP | dl-THP |
| 0.1 | mg/kg | 26.82 ± 4.38 | 28.92 ± 4.73 | 11.55 ± 2.50 | 8.77 ± 1.88 | 42.83 ± 4.26* | 30.58 ± 5.83* |
| 0.5 | mg/kg | 27.92 ± 4.08 | 30.25 ± 5.55 | 11.75 ± 3.14 | 13.15 ± 3.33 | 41.82 ± 6.37 | 43.13 ± 4.17 |
| 1.0 | mg/kg | 26.92 ± 3.61 | 31.20 ± 4.16 | 12.00 ± 2.00 | 16.00 ± 3.13 | 44.47 ± 3.10* | 51.17 ± 5.48* |
| 5.0 | mg/kg | 11.17 ± 5.13** | 30.15 ± 4.83** | 3.33 ± 1.92** | 15.23 ± 3.19** | 29.63 ± 5.03** | 50.21 ± 4.04** |

| | | | | |
|---|---|---|---|---|
| DZ 1 | mg/kg | 38.25 ± 7.06 | 21.50 ± 4.52 | 55.42 ± 4.10 |

| | | | |
|---|---|---|---|
| Control | 26.43 ± 3.41 | 8.00 ± 1.84 | 30.02 ± 4.24 |

Comparison with same dosage of l-THP & dl-THP
*p < 0.05;
**p < 0.01
n = 12

The results in Table 6 compare the time spent and percentage of open arm entry of mice between l-THP and dl-THP in the elevated plus-maze test. Both l-THP & dl-THP increases the time spent and percentage of open arm entry compared to normal control group, p<0.01. The most medicinal dosage is 1 mg/kg for l-THP and 5 mg/kg for dl-THP. For both of l-THP & dl-THP groups, the time spent and percentage of open arm entry are less than DZ 1 mg/kg group. For the l-THP 5 mg/kg group, the time spent and percentage of open arm entries are significantly less than normal control group and most of the mice fell asleep in the closed arm, representing the hypnotic effect at this dosage. No statistic difference exists between the l-THP & dl-THP groups except for the 0.1 mg/kg group, p<0.05 and the 5 mg/kg group, p<0.01.

TABLE 6

| | | Time spent | | % | |
|---|---|---|---|---|---|
| Group | | l-THP | dl-THP | l-THP | dl-THP |
| 0.5 | mg/kg | 71.75 ± 21.08 | 80.42 ± 15.23 | 25.81 ± 7.09 | 29.25 ± 5.10 |
| 1.0 | mg/kg | 80.08 ± 21.03 | 88.40 ± 17.37 | 28.06 ± 6.70 | 29.95 ± 5.31 |
| 5.0 | mg/kg | 32.33 ± 14.43** | 95.00 ± 23.56** | 11.46 ± 5.48** | 32.79 ± 8.60** |

| | | | |
|---|---|---|---|
| DZ 1 | mg/kg | 131.93 ± 19.42** | 45.19 ± 4.53** |

| | | |
|---|---|---|
| Control | 52.07 ± 8.74 | 18.61 ± 3.46 |

Comparison with same dosage of l-THP & dl-THP
*p < 0.05;
**p < 0.01
n = 12

Hole-Board Test

The hole-board apparatus is a 0.5 cm thick walled wood arena of 60×60×30 cm, with four 5 cm equidistant 4 cm diameter holes spaced on the floor. The mice are placed on the centre of the arena and the number of head-dip on the hole and the number of rearing are counted during a 300 second test period (File S. E. et al., “The effects of triazolobenzo-diazepines in two animal tests of anxiety and in the holeboard.”, Br J Pharmacol. 1985 November; 86(3): 729-35; PMID: 2866006). After each trial, the floor of the apparatus was wiped and dried thoroughly with tissue to remove traces of the previous path. A decrease of the two parameters compared with the normal control group reveals a sedative behaviour.

Table 7 shows the number of head-dip and rearing of mice after oral administration of l-THP in the hole-board test. Except 5.0 mg/kg group, the number of head-dip for dosages of l-THP are greater than the normal control group, p<0.05. There are no significant differences in the number of rearing between the said groups. The number of head-dip and rearing of 5.0 mg/kg group are significantly less than the normal control group, p<0.05.

TABLE 7

| Group | | Total No. | Head-dip No. | Rearing No. |
|---|---|---|---|---|
| 0.05 | mg/kg | 42.67 ± 6.64* | 15.83 ± 3.01* | 26.83 ± 5.51 |
| 0.1 | mg/kg | 39.09 ± 5.63* | 18.73 ± 6.56* | 20.36 ± 9.54 |
| 0.5 | mg/kg | 41.00 ± 10.20 | 15.18 ± 11.77* | 27.08 ± 13.64 |
| 1.0 | mg/kg | 40.33 ± 11.00 | 16.83 ± 10.20* | 23.50 ± 9.27 |
| 2.5 | mg/kg | 38.08 ± 6.43 | 15.67 ± 4.00 | 22.42 ± 4.06 |
| 5.0 | mg/kg | 22.92 ± 11.35* | 8.75 ± 5.56 | 14.17 ± 6.60* |
| DZ 1 | mg/kg | 42.00 ± 7.11 | 15.62 ± 5.82* | 26.38 ± 8.51 |

| | | | |
|---|---|---|---|
| Control | 35.71 ± 7.37 | 10.89 ± 5.56 | 24.93 ± 9.49 |

*p < 0.05
n = 12

Table 8 shows the comparison of head-dip and rearing of mice between l-THP and dl-THP of same dosages in the hole-board test. The total number of head-dip and the number of rearing of 1 mg/kg l-THP are significantly greater than 1 mg/kg dl-THP, p<0.05. Alternatively, the total number of head-dip and the number of rearing of 5 mg/kg l-THP are significantly less than 5 mg/kg dl-THP, p<0.01. There is no significant statistic difference for other dosage groups. The activities of 5 mg/kg l-THP group are much less than 5 mg/kg dl-THP group and the normal control group. There is no significant difference between the best medicinal dosage of l-THP/dl-THP groups and 1 mg/kg DZ group, p<0.05.

TABLE 8

| | | Total no. | | No. of head-dip | | No. of rearing | |
|---|---|---|---|---|---|---|---|
| Group | | l-THP | dl-THP | l-THP | dl-THP | l-THP | dl-THP |
| 0.1 | mg/kg | 39.09 ± 5.63 | 35.58 ± 16.47 | 18.73 ± 6.56* | 13.42 ± 12.06 | 20.36 ± 9.54 | 22.17 ± 12.76 |
| 0.5 | mg/kg | 41.00 ± 10.20 | 44.17 ± 13.53 | 15.18 ± 11.77 | 17.67 ± 11.14 | 27.08 ± 13.64 | 26.50 ± 10.06 |
| 1.0 | mg/kg | 40.33 ± 11.00* | 33.60 ± 20.37* | 16.83 ± 10.20 | 13.20 ± 10.64 | 23.50 ± 9.27 | 20.40 ± 15.74 |
| 5.0 | mg/kg | 22.92 ± 11.35** | 37.46 ± 15.95** | 8.75 ± 5.56 | 11.08 ± 6.34 | 14.17 ± 6.60** | 26.39 ± 12.28** |

| | | | | |
|---|---|---|---|---|
| DZ 1 | mg/kg | 42.00 ± 7.11 | 15.62 ± 5.82 | 26.38 ± 8.51 |

| | | | |
|---|---|---|---|
| Control | 35.71 ± 7.37 | 10.89 ± 5.56 | 24.93 ± 9.49 |

Comparison with same dosage of l-THP & dl-THP
*p < 0.05;
**p < 0.01;
n = 12

The contents of each of the references discussed herein, including the references cited therein, are herein incorporated by reference in their entirety. Where “PMID” reference numbers are given for publications, these are the PubMed identification numbers allocated to them by the US National Library of Medicine.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating anxiety disorder in a patient in need thereof comprising administering to said patient a therapeutically effective amount of l-THP.

2. A method according to claim 1 wherein said method of treating an anxiety disorder further includes the step of maintaining a muscular relaxation state at a pre-treatment level.

3. A method of anesthetizing a patient in need thereof comprising administering to said patient a therapeutic amount of l-THP.
